(19) United States
(12) Patent Application Publication
(10) Pub. No.: US 2004/0131653 A1
(43) Pub. Date: Jul. 8, 2004

(54) Vasodilator impregnated devices and methods

(76) Inventors: Ramanan Ramaswami, Burgess Hill (GB); Ganesh Ramaswami, New York, NY (US)

Correspondence Address: DUANE MORRIS LLP, Suite 100, 100 College Road West, Princeton, NJ, 08540, US

(21) Appl. No.: 10/741,830

(22) Filed: Dec. 19, 2003

Related U.S. Application Data
(63) Continuation of application No. 10/081,711, filed on Feb. 22, 2002.
(60) Provisional application No. 60/271,018, filed on Feb. 22, 2001.

(51) Int. Cl. A61K 31/554; A61K 31/21

(52) U.S. Cl. 424/426; 514/355; 514/211.07; 514/509

(57) ABSTRACT

A device and method is described for providing sustained vasodilation at a site in a patient. The device includes a biocompatible and possibly biodegradable carrier, and a vasodilator incorporated into the carrier. The vasodilator is present in a topically effective amount to achieve sustained vasodilation at a selected site in a patient. The vasodilator may be nitroglycerine or calcium channel blockers. The carrier includes methylcellulose and equine collagen, and may be in the form of a strip. The method includes administering, at a selected site in a patient, a vasodilator incorporated into a carrier. The vasodilator is present in a topically effective amount to achieve sustained vasodilation at the selected site.

### CLAIM FOR PRIORITY

This application is a continuation of U.S. patent application Ser. No. 10/081,711, filed Feb. 22, 2002.

### BACKGROUND OF THE INVENTION

1. Field of the Invention

The present invention relates generally to vasodilator devices, and more particular, relates to a vasodilator incorporated into a biocompatible material.

2. Description of the Related Art

Atherosclerosis which occurs in the process of aging, leads to narrowing of the vessel lumen. The result of decreased blood flow results in end-organ damage. When flow to the heart is decreased, the patient develops symptoms of angina; similarly, a decreased flow to the brain results in stroke. When flow to the intestines is decreased, the patient develops symptoms of intestinal angina. A reduction in flow to the legs results in symptoms of claudication, rest pain and gangrene.

The established ‘gold-standard’ for the treatment of long-segment occlusive arterial disease is ‘bypass grafting’ which utilizes autologuous arterial or venous grafts (as conduits) to ‘bypass’ the occluded segment of artery. An estimated 330,000 coronary bypass procedures and 180,000 peripheral artery revascularizations are performed annually in the United States alone.

Grafts used in coronary artery bypass surgery may be either arterial (internal mammary, radial or less frequently gastroepiploic) or reversed vein (usually saphenous). It is well established that the best results in peripheral bypass surgery for limb-threatening ischemia are achieved with saphenous vein grafts. Vein grafts are also used in other situations, with the internal iliac vein being used to bypass the occluded portal vein for the treatment of portal hypertension, or during liver transplant, and vein patches are frequently used for increasing the diameter of the carotid artery during carotid endarterectomy.

Vasospasm occurring in coronary grafts has been shown to be associated with hemodynamic instability and myocardial infarction. Under normal hemodynamic conditions the flow through an artery is laminar and is governed by the systemic arterial blood pressure and the resistance imposed by the vessel. The resistance to flow offered by a graft is related to the viscosity of the blood, the length and radius of the artery and the resistance of the distal vascular bed. Given that the viscosity, length of the graft, and vascular resistance of the distal arterial bed cannot be easily manipulated, it is still possible to increase the radius of the vessel in an attempt to increase flow. Since the resistance to flow is inversely proportional to the fourth power of the radius, dilating the graft will produce a major increase in flow.

A major problem faced by surgeons involved in performing bypass procedures is the propensity of arterial and venous grafts to go into vasospasm. Although this is more marked in arterial grafts because of their greater content of smooth muscle, vein graft spasm also poses a formidable problem.

Various pharmacological and physical methods have been proposed to overcome perioperative spasm of grafts. Probing of arteries (internal thoracic) in the experimental dog causes endothelial cell loss and impaired release of prostacycline and endothelium-derived relaxing factor. Based on this observation, probing of arterial grafts is not typically performed by surgeons.

Hydrostatic dilation of the arteries has been shown to greatly improve the blood flow through the artery. Such dilatation in the porcine model, however, has been shown to produce disruption and fenestration of the internal elastic lamina and functional damage to the media. A direct consequence of this may be a decreased capacity to generate adenosine, a potent vasodilator and antiplatelet aggregating factor. Additionally, injury to the media which is known to stimulate smooth muscle cell proliferation fundamental to the development of intimal hyperplasia in saphenous vein grafts leads to graft failure.

A variety of agents have been used to increase flow in autogenous grafts. This can be achieved either with topical application of vasodilators such as Nitroprusside, intraluminal injection of Papaverine, or hydrostatic dilatation with Papaverine, for example. Vasospasm is typically treated in the peri-operative period by the use of vasodilators such as papaverine, or nitroglycerine, which is sprayed on the surface of the graft, prior to closing the wound.

A study of the effect of topical vasodilators used to overcome vasospasm showed that although papaverine caused a modest 70% increase in free flow, it was the least effective of the agents investigated. Calcium channel blockers and nitroglycerine raised flow by 160%. The beneficial effect of topical vasodilators is, however, only temporary and may not prevent postoperative spasm of the grafts.

Persistent vasospasm in the post-operative period, however, frequently leads to graft thrombosis, secondary to diminution of blood flow. The first few post-operative days are therefore critical in terms of graft patency and development of collaterals. Graft thrombosis frequently necessitates re-operation which poses additional risks to the patient and also increased spending of health care dollars.

For example, graft thrombosis after coronary grafting may lead to recurrence of symptoms of myocardial ischemia and death. After peripheral revascularization, thrombosed grafts frequently necessitate re-operation for limb salvage. Also, vasospasm in the grafted hepatic artery/vein or renal artery/vein, in transplant patients, leads to graft thrombosis, jeopardizing the fate of the transplanted liver or kidney.

In spite of the major advances in bypass grafting in recent years, no single method has been effectively developed to overcome the problem of vasospasm in the immediate post-operative period. Although vasodilators may be effectively used in the peri/intra-operative period to spray and dilate the graft, direct access to the graft requiring re-operation is not desired in the post-operative period.

Accordingly, there still remains a need to overcome the problems associated with vasospasm in the post-operative period.

### SUMMARY OF THE INVENTION

Briefly described, a device according to an aspect of the invention provides sustained vasodilation at a selected site in a patient. The device includes a biocompatible carrier, and a vasodilator incorporated into the carrier. The carrier may also be biodegradable. The vasodilator is present in a topically effective amount to achieve sustained vasodilation at a selected site in a patient. The vasodilator is selected from the nitroglycerine and calcium channel blockers. Calcium channel blockers include verapamil, diltiazem, and nifedipine. If the vasodilator used is nitroglycerine, a concentration of about 1 mg/ml of solution is incorporated into the carrier. If the vasodilator used is a calcium channel blocker, such as verapamil, a concentration of about 5 mg/2 ml of solution is incorporated into the carrier. The carrier material includes methylcellulose and equine collagen, and may be in the form of a strip. The device may be disposed in a sterile container. Vasodilation may be sustained for several days.

The method according to an aspect of the invention includes administering, at a selected site in a patient, a vasodilator incorporated into a carrier, where the vasodilator is present in a topically effective amount to achieve sustained vasodilation at the selected site.

### BRIEF DESCRIPTION OF THE DRAWINGS

FIG. 1 is a perspective view of the device according to an aspect of the invention; and

FIG. 2 is a perspective view of the device (in phantom) disposed in a sterile container.

### DETAILED DESCRIPTION OF THE INVENTION

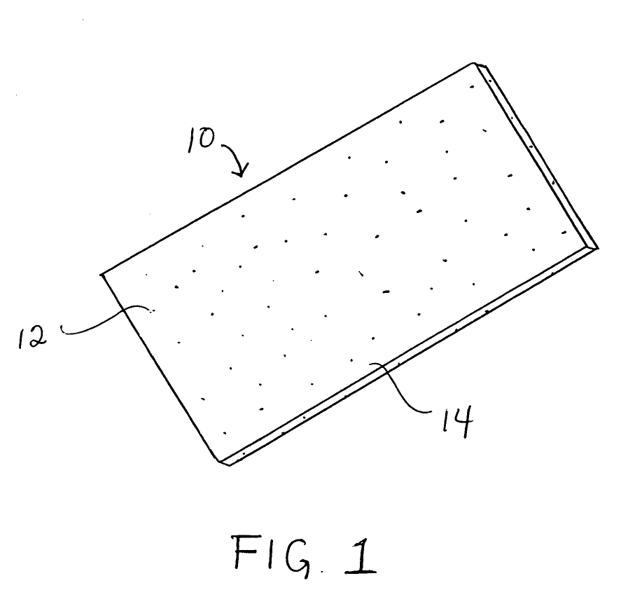

Referring to FIG. 1, a device 10 according to an aspect of the invention is illustrated in the form of a strip. A vasodilating agent 12 is illustrated as impregnated into a carrier material 14. The device 10 may be wrapped about a graft, or, alternatively, placed over or adjacent to a graft. In the peri-operative period, the device 10 is wrapped about or laid over an anastomosis (proximal or distal) venous or arterial graft site, or disposed at a selected location where a vasodilator effect is necessary or desired to avoid vasospasm.

The consistency of the carrier 14 is soft and malleable, which enables it to be wrapped circumferentially around an arterial or venous graft. The carrier material 14 is composed of a material that is inert and biocompatible, without producing any local inflammatory response in the graft wall. The carrier material 14 may biodegrade over a period of about a week to about two weeks, for possibly up to one month. A preferred carrier material is methylcellulose. Methylcellulose is available from Ethicon, Inc. in Somerville, N.J., and is sold under the trademark SURGICEL® Absorbable Haemostat. An alternative preferred carrier material is Tissue Fleece, available from Baxter Healthcare Ltd, in Berkshire, England. Tissue Fleece is equine collagen which is typically sold, in strip form, in convenient sterile packs. The strips, when moistened in saline before use, are soft and malleable, and may be laid on a conduit without causing kinks in the graft, particularly after the wound is closed. When disposed onto a graft, the collagen strip conforms and molds to the shape thereof, and biodegrades over several days. Methylcellulose also conforms and molds to the shape of the graft. Other suitable inert carrier materials that are biocompatible may also be used.

Suitable dimensions for the material 14 in the form of a strip are about 3 cm.×5 cm. It is to be understood that the material 14, although illustrated in the form of a strip, may take any shape, and may have different dimensions, depending upon the needs of the surgeon, the patient, or the application desired. The term “patient” refers to a mammal, and includes both humans and animals. The dimensions selected should be suitable for circumferentially placing the device 10 around a graft, or for encompassing an area over or adjacent to a graft. If the material 14 used is greater in dimension than about 3 cm.×5 cm., a proportionate increase in the amount of vasodilator agent may be used relative to the size of the carrier material 14.

In the present invention, the biocompatible carrier material 14 is impregnated or saturated with an effective amount of a pharmaceutically and topically acceptable vasodilator 12. The vasodilator 12, in liquid form, may be drawn into a syringe, or other suitable instrument, and then squirted onto the carrier material 14. The vasodilator 12 incorporated into the carrier material 14 is selected from calcium channel blockers, nitroglycerine, and other pharmaceutically and topically acceptable vasodilator agents. An effective amount includes an amount necessary to achieve sustained release of the vasodilator for several days, for up to a week or longer.

Suitable calcium channel blockers for use in the present invention include verapamil, diltiazem, and nifedipine, which are known and available from a number of sources. Although nifedipine is not presently commercially available as an IV (intravenous) preparation, a manufacturer can prepare nifedipine in that form. Nitroglycerine is also known, and is available from a number of sources. Nitroglycerine is presently available as a 50 mg/50 ml solution which is suitable for use in the present invention.

If the vasodilator 12 selected is a calcium channel blocker, such as verapamil, an effective concentration is about 5 mg/2 ml of solution, for each 3 cm.×5 cm. strip of material 14. If the vasodilator 12 selected is nitroglycerine, an effective concentration is about 1 mg/ml of solution, for each 3 cm.×5 cm. strip of material 14. Higher concentrations of vasodilator agents may also be used without causing toxicity to a patient, since the effect of the vasodilator agent is topical and not systemic.

By disposing the device 10 about or adjacent an arterial or venous graft, the carrier 14 releases the vasodilator 12 into the wall of the artery or vein graft at a steady rate which lasts for several days, up to a week or possibly longer, depending upon the amount and concentration of the vasodilator 12 used. The carrier material 14 may also affect the rate of release. The sustained release of the vasodilator locally into the graft wall results in sustained vasodilatation of the graft wall with concomitant increased blood flow, and also avoids problems associated with the systemic release of vasodilators. The sustained vasodilatation of arterial and venous grafts prevents vasospasm in the immediate postoperative period, and tends to decrease the need for re-intervention for thrombosed grafts, secondary to vasospasm with concomitant low flow, which is beneficial to the patient.

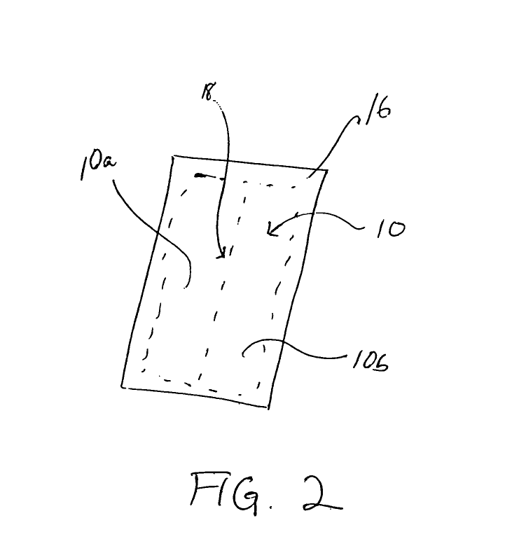

The device 10 may be disposed in a sterile container or package 16, which may be available off the shelf, easily opened, and ready for use by a surgeon or other medical personnel, as illustrated in FIG. 2. Other embodiments of the package 16 are also possible. The device 10 may also include perforations 18 (shown in phantom) along the length thereof, which allows the surgeon or other medical personnel to remove a portion 10a of the device 10 for use at a selected site, and use the remaining portion 10b at another selected site in the patient.

Although the invention has been shown and described in a preferred form with a certain degree of particularity, it is to be understood by those skilled in the art that the present disclosure has been made only by way of example, and that numerous modifications may be made without departing from the spirit and scope of the invention as hereinafter claimed. It is intended that the patent shall cover by suitable expression in the appended claims whatever features of patentable novelty exist in the invention disclosed.

The present invention shall be described in further detail by reference to the following example which is provided for illustrative purposes only, and is not intended to be limiting.

### EXAMPLE 1

One (1) cm. long segments of internal mammary artery (IMA), radial artery or saphenous vein harvested during surgery were cut into three (3) mm conduit rings and placed in an organ bath composed of Kreb's solution. A standard Kreb's solution has the following composition: Na⁺ 144, K⁺ 5.9, Ca⁺⁺ 2.5, Mg⁺⁺ 1.2, Cl⁻ 128, HCO₃⁻ 25, SO₄⁻⁻ 1.2, H₂PO₄⁻ 1.2 and glucose 11 mmol/L. The harvested 3 mm conduit rings were placed in 100 ml of the Kreb's solution in a container maintained at 37° C. and continuously aerated with 95% O₂ and 5% CO₂. Measurement of the conduit was made 30 minutes after placement in the bath to overcome any vasospasm. Calipers were used and baseline measurements were made of the diameter of the conduit. The conduit placed in Kreb's solution alone had measurements at six time points namely 0 hours (baseline), 2 hours, 12 hours, 24 hours, 48 hours and 72 hours. The maximum % change in diameter from the given time points in comparison to baseline (0 hour) were used to calculate the diameter augmentation (%).

Two vasodilator agents, Verapamil (Ca⁺⁺ channel blocking agent) and nitroglycerine (NTG) were used to study the increase in diameter (augmentation) compared to baseline. The Ca⁺⁺ channel-blocking agent Verapamil was available as a 5 mg in 2 ml preparation and the vasodilatory effect of 100 nanomol/L was studied for the purpose of the experiment. Therefore 4.9 microgram (or 0.00196 ml of injectable solution) was added to 100 ml of Kreb's solution containing the conduit. Nitroglycerine (NTG) was available as a 50 mg in 50 ml solution and 0.5 micromol/L (0.0116 ml) was added to 100 ml of Krebs solution containing the conduit.

Table I illustrates the diameter augmentation of conduits after vasodilation with verapamil and NTG in comparison to conduits placed in Kreb's solution alone which acted as control. For the arterial conduit (IMA) the diameter augmentation after placement in Kreb's solution alone was modest ranging from 13% to 27%; with NTG, the augmentation increased from 47-73% and with verapamil the increases ranged from 59-64%. With the radial arterial graft the augmentation with Kreb's solution alone was minimal (8-9%); with NTG, the increase was 42-52% and for verapamil 52-62%. When vein conduits were studied the augmentation in diameter with Kreb's solution ranged from 17-28%; for NTG, the diameter augmentation was 51-66% and for verapamil 66-70%.

TABLE I

| | | 0 | 2 | 12 | 24 | 48 | 72 | Aug |
|---|---|---|---|---|---|---|---|---|
| Conduit | Agent | hrs. | hrs. | hrs. | hrs. | hrs. | hrs. | % |
| IMA | Kreb's soln. | 1.5 | 1.7 | 1.8 | 1.7 | 1.8 | 1.8 | 20% |
| IMA | Kreb's soln. | 2.3 | 2.5 | 2.3 | 2.7 | 2.6 | 2.6 | 13% |
| IMA | Kreb's soln. | 1.8 | 2.1 | 2.0 | 2.1 | 2.2 | 2.3 | 27% |
| IMA | NTG | 1.5 | 2.1 | 2.3 | 2.3 | 2.4 | 2.9 | 73% |
| IMA | NTG | 2.2 | 2.4 | 2.5 | 2.6 | 2.8 | 3.1 | 50% |
| IMA | NTG | 2.3 | 2.7 | 2.8 | 2.6 | 2.9 | 3.4 | 47% |
| IMA | verapamil | 1.8 | 2.4 | 2.6 | 2.5 | 3.0 | 2.9 | 61% |
| IMA | verapamil | 1.7 | 2.1 | 2.4 | 2.3 | 2.5 | 2.7 | 59% |
| IMA | verapamil | 1.9 | 2.7 | 2.8 | 2.9 | 3.0 | 3.1 | 64% |
| Radial | Kreb's soln. | 2.4 | 2.6 | 2.7 | 2.5 | 2.5 | 2.6 | 8% |
| Radial | Kreb's soln. | 2.1 | 2.4 | 2.4 | 2.3 | 2.3 | 2.3 | 9% |
| Radial | Kreb's soln. | 2.3 | 2.5 | 2.6 | 2.4 | 2.5 | 2.5 | 8% |
| Radial | NTG | 2.5 | 2.9 | 3.6 | 3.8 | 3.7 | 3.8 | 52% |
| Radial | NTG | 2.3 | 3.0 | 3.0 | 2.9 | | 3.3 | 44% |
| Radial | NTG | 2.6 | 3.3 | 3.5 | 3.7 | | 3.7 | 42% |
| Radial | verapamil | 2.3 | 3.5 | 3.5 | | 3.6 | 3.6 | 57% |
| Radial | verapamil | 2.4 | 3.9 | | 3.8 | | 3.9 | 62% |
| Radial | verapamil | 2.7 | 4.1 | 3.9 | | 3.8 | 3.9 | 52% |
| Vein | Kreb's soln. | 2.5 | 3.0 | 2.9 | 3.0 | 2.8 | 3.0 | 20% |
| Vein | Kreb's soln. | 3.5 | 4.5 | 4.3 | 4.5 | 4.4 | 4.2 | 28% |
| Vein | Kreb's soln. | 2.3 | 2.5 | 2.6 | 2.4 | 2.7 | 2.4 | 17% |
| Vein | NTG | 2.4 | 4.0 | 3.8 | 4.1 | 3.7 | 3.7 | 66% |
| Vein | NTG | 3.7 | 6.2 | 6.0 | 5.9 | 6.1 | 6.2 | 67% |
| Vein | NTG | 2.7 | 4.0 | 4.1 | 3.8 | 3.9 | 3.7 | 51% |
| Vein | verapamil | 3.0 | 5.1 | 4.8 | 5.0 | 4.7 | 4.8 | 70% |
| Vein | verapamil | 3.5 | 5.2 | 5.7 | 5.8 | 5.7 | 5.8 | 65% |
| Vein | verapamil | 3.3 | 5.5 | 5.3 | 5.2 | 5.5 | 5.4 | 66% |

In Table I, the term “Aug %” refers to the maximum increase in diameter at a given time point compared to baseline (0 hrs.) The acronym for the conduit “IMA” refers to internal mammary artery. The acronym for the agent “NTG” refers to nitroglycerine. The agent verapamil is a calcium channel blocker.

Example 1 demonstrates that the diameter augmentation of conduits (arterial and venous) following the addition of vasodilatory agents (verapamil and NTG) is greater than when conduits were placed in Kreb's solution alone (control).

What is claimed is:

1. A biodegradable device for providing sustained vasodilation at a graft site within a patient, comprising:
a biocompatible, biodegradable carrier sized to circumferentially encompass said site;
a vasodilator incorporated into said carrier, which vasodilator is present in a topically effective amount to achieve sustained vasodilation at said site in a patient.

2. The device of claim 1, wherein said vasodilator is selected from the group consisting of nitroglycerine and calcium channel blockers.

3. The device of claim 2, wherein said calcium channel blockers are selected from the group consisting of: verapamil, diltiazem, and nifedipine.

4. The device of claim 1, wherein said vasodilator comprises a calcium channel blocker and is incorporated into said carrier at a concentration of about 5 mg/2 ml of solution.

5. The device of claim 4, wherein said calcium channel blocker is verapamil.

6. The device of claim 1, wherein said vasodilator is nitroglycerine and is incorporated into said carrier at a concentration of about 1 mg/ml of solution.

7. The device of claim 1, wherein said carrier comprises methylcellulose.

8. The device of claim 1, wherein said carrier comprises equine collagen.

9. The device of claim 1, wherein said carrier is in the form of a strip.

10. The device of claim 1, wherein said device is disposed in a sterile container.

11. The device of claim 1, wherein said vasodilation is sustained for several days.

12. A method for providing sustained vasodilation at a selected graft site within a patient, comprising:
administering, at said selected site in a patient, a vasodilator incorporated into a biocompatible, biodegradable carrier sized to circumferentially encompass said site, which vasodilator is present in a topically effective amount to achieve sustained vasodilation at said selected site.

13. The method of claim 12, wherein said vasodilator is selected from the group consisting of nitroglycerine and calcium channel blockers.

14. The method of claim 13, wherein said calcium channel blockers are selected from the group consisting of: verapamil, diltiazem, and nifedipine.

15. The method of claim 12, wherein said vasodilator is a calcium channel blocker and is incorporated into said carrier at a concentration of about 5 mg/2 ml of solution.

16. The method of claim 15, wherein said calcium channel blocker is verapamil.

17. The method of claim 12, wherein said vasodilator comprises nitroglycerine, and is incorporated into said carrier at a concentration of about 1 mg/ml of solution.

18. The method of claim 12, wherein said carrier comprises methylcellulose.

19. The method of claim 12, wherein said carrier comprises equine collagen.

20. The method of claim 12, wherein said carrier is in the form of a strip.

21. The method of claim 12, wherein said vasodilation is sustained for several days.

22. The device of claim 1, wherein said carrier is perforated.

23. The method of claim 12, wherein said carrier is perforated.
